(19) United States
(12) Patent Application Publication
(10) Pub. No.: US 2004/0024274 A1
(43) Pub. Date: Feb. 5, 2004

(54) Method for the hydrogenation of unsubstituted or alkyl substituted aromatics using a catalyst with a structured or monolithic support

(76) Inventors: Arnd Böttcher, Frankenthal (DE); Jochem Henkelmann, Mannheim (DE); Mathias Haake, Mannheim (DE); Gerd Kaibel, Lampertheim (DE)

Correspondence Address: KEIL & WEINKAUF, 1350 CONNECTICUT AVENUE, N.W., WASHINGTON, DC, 20036, US

(21) Appl. No.: 10/398,177

(22) PCT Filed: Oct. 9, 2001
(86) PCT No.: PCT/EP01/11649

(51) Int. Cl. C07C 5/10; B01J 23/00

(52) U.S. Cl. 585/266; 502/325; 585/268; 585/275

(57) ABSTRACT

A process for the hydrogenation of a monocyclic or polycyclic aromatic which may be unsubstituted or substituted by at least one aikyi group comprises bringing the aromatic into contact with a hydrogen-containing gas in the presence of a catalyst comprising at least one metal of transition group VIII of the Periodic Table as active metal applied to a structured or monolithic support.

The present invention relates to a process for the hydrogenation of monocyclic or polycyclic aromatics which may be unsubstituted or substituted by at least one alkyl group to form the corresponding cycloaliphatics, in particular of benzene to cyclohexane, by bringing the aromatic into contact with a hydrogen-containing gas in the presence of a catalyst comprising at least one metal of transition group VIII of the Periodic Table as active metal applied to a structured or monolithic support.

There are numerous processes for the hydrogenation of, for example, benzene to cyclohexane. These hydrogenations are predominantly carried out in the gas or liquid phase over particulate nickel and platinum catalysts (cf., for example, U.S. Pat No. 3,597,489, GB 1 444499 and GB 992 104). Typically, the major part of the benzene is firstly hydrogenated to cyclohexane in a main reactor and the conversion to cyclohexane is subsequently completed in one or more after-reactors.

The strongly exothermic hydrogenation reaction requires careful control of pressure, temperature and residence time to achieve complete conversion at high selectivity. In particular, significant formation of methylcyclopentane, which is favored at higher temperatures, has to be suppressed. Typical cyclohexane specifications require a residual benzene content of<100 ppm and a methylcyclopentane content of<200 ppm. The content of n-paraffins (n-hexane, n-pentane, etc.) is also critical. The formation of these undesired compounds is likewise favored at relatively high hydrogenation temperatures and, like methylcyclopentane, they can be separated from the cyclohexane produced only by complicated separation operations. The separation can be carried out, for example, by extraction, rectification or by use of molecular sieves as described in GB 1 341 057. The catalyst used for the hydrogenation also has a strong influence on the extent of undesirable methylcyclopentane formation.

In view of this background, it is desirable to carry out the hydrogenation at the lowest possible temperatures. However, this is restricted by the fact that, depending on the type of hydrogenation catalyst used, the catalyst displays a sufficiently high hydrogenation activity capable of giving economically acceptable space-time yields only above a relatively high temperature.

The nickel and platinum catalysts used for the hydrogenation of benzene have a series of disadvantages. Nickel catalysts are very sensitive to sulfur-containing impurities in the benzene, so that either very pure benzene has to be used for the hydrogenation or, as described in GB 1 104 275, a platinum catalyst which tolerates a higher sulfur content is used in the main reactor and thus protects the after-reactor which is charged with a nickel catalyst. Further possibilities are doping the catalyst with rhenium (GB 1 155 539) or producing the catalyst using ion exchangers (GB 1 144 499). However, the production of such catalysts is complicated and expensive. The hydrogenation can also be carried out over Raney nickel (U.S. Pat No. 3,202,723), but a disadvantage of this is the ready combustibility of this catalyst. Homogeneous nickel catalysts can also be used for the hydrogenation (EP 0 668 257). However, these catalysts are very water-sensitive, so that the benzene used firstly has to be dried to a residual water content of<1 ppm in a drying column prior to the hydrogenation. A further disadvantage of the homogeneous catalyst is that it cannot be regenerated.

Platinum catalysts have fewer disadvantages than nickel catalysts, but are much more expensive to produce. Very high hydrogenation temperatures are necessary both when using platinum catalysts and when using nickel catalysts, which can lead to significant formation of undesirable by-products.

The hydrogenation of benzene to cyclohexane over ruthenium catalysts is not carried out industrially, but the patent literature does refer to the use of ruthenium-containing catalysts for this application.

According to SU 319 582, suspended Ru catalysts doped with Pd, Pt or Rh are used for preparing cyclohexane from benzene. However, the catalysts are very expensive because of the use of Pd, Pt or Rh. Furthermore, the work-up and recovery of the catalyst is complicated and expensive in the case of suspended catalysts.

According to SU 403 658, a Cr-doped Ru catalyst is used for preparing cyclohexane. The active metals are supported on Al₂O₃ granules. The hydrogenation is carried out at from 160 to 180° C., resulting in formation of a significant amount of undesirable by-products.

U.S. Pat No. 3,917,540 discloses Al₂O₃-supported catalysts for preparing cyclohexane. These comprise a noble metal from transition group VIII of the Periodic Table as active metal, also an alkali metal plus technetium or rhenium. The Al₂O₃ supports are in the form of spheres, granules or the like. A disadvantage of such catalysts is that a selectivity of only 99.5% is achieved.

Finally, U.S. Pat No. 3,244,644 describes ruthenium hydrogenation catalysts supported on η-Al₂O₃ which are said to be suitable for the hydrogenation of benzene. These catalysts are shaped as pellets of not more than ¼ inch and contain at least 5% of active metal; the preparation of η-Al₂O₃ is complicated and expensive.

Apart from the above-described particulate catalysts or suspended catalysts, monolithic supported catalysts in the form of ordered packing with catalytically active layers which can be used for hydrogenation reactions are also known from the prior art.

EP 0 564 830 B1 describes, for example, a monolithic supported catalyst which can comprise elements of group VIII of the Periodic Table as active components.

EP 0 803 488 A2 discloses a process for the reaction, for example the hydrogenation, of an aromatic compound bearing at least one hydroxyl group or amino group on an aromatic ring in the presence of a catalyst comprising a homogeneous ruthenium compound which has been deposited in situ on a support, for example a monolith. The hydrogenation is carried out at pressure of more than 50 bar and temperatures of preferably from 150° C. to 220° C.

It is an object of the present invention to provide an economical process for the hydrogenation of monocyclic or polycyclic aromatics which may be unsubstituted or substituted by at least one alkyl group to form the corresponding cycloaliphatics, in particular of benzene to cyclohexane.

We have found that this object is achieved by the process of the present invention for the hydrogenation of at least one monocyclic or polycyclic aromatic which may be unsubstituted or substituted by at least one alkyl group by bringing the aromatic into contact with a hydrogen-containing gas in the presence of a catalyst comprising at least one metal of transition group VIII of the Periodic Table as active metal applied to a structured or monolithic support.

It has surprisingly been found that such aromatics can be hydrogenated selectively and at high space-time yields to give the corresponding cycloaliphatics over catalysts having a structured or monolithic support even at pressures and temperatures significantly lower than those in processes of the prior art. This was very surprising because even the hydrogenation of aromatics having polar substituents, as described in EP 0 803 488 A2, which have a significantly higher reactivity than monocyclic or polycyclic aromatics which are unsubstituted or substituted by at least one alkyl group, requires very high pressures and temperatures. From this it was not to be expected that such aromatics would be able to be hydrogenated in an economical manner by means of the process of the present invention. At the low pressures and temperatures which can be used according to the present invention, the formation of undesirable by-products such as methylcyclopentane or other n-paraffins is virtually nonexistent, so that complicated purification of the cycloaliphatics produced becomes unnecessary, which makes the process very economical.

In the context of the present invention, structured supports are supports which have a regular two-dimensional or three-dimensional structure and are in this way distinguished from particulate catalysts which are used as a loose, random bed. Examples of structured supports are supports made up of threads or wires, usually in the form of support sheets such as woven fabrics or meshes, knitteds or felts. Structured supports can also be films, foils or metal sheets which may also have recesses or holes, for example perforated metal sheets or expanded metal. Such essentially two-dimensional structured supports can, for the present use, be shaped to produce appropriately shaped three-dimensional structures, referred to as monoliths or monolithic supports, which can in turn be used, for example, as catalyst packing or column packing. Such packing can consist of a plurality of monoliths. It is likewise possible for the monoliths not to be built up from two-dimensional support sheets but for them to be produced directly without intermediate stages, for example the ceramic monoliths with flow channels known to those skilled in the art.

As structured supports, it is possible to use two-dimensional structured supports such as woven fabrics or meshes, knitteds, felts, films and foils, metal sheets, e.g. perforated metal sheets, or expanded metals. However, essentially three-dimensional structures such as monoliths can also be used.

The structured supports or monoliths can comprise metallic, inorganic, organic or synthetic materials or combinations of such materials.

Examples of metallic materials are pure metals such as iron, copper, nickel, silver, aluminum and titanium or alloys such as steels, for instance nickel steel, chromium steel and molybdenum steel, brass, phosphor bronze, Monell and nickel silver. Examples of ceramic materials are aluminum oxide, silicon dioxide, zirconium dioxide, cordierite and steatite. It is also possible to use carbon.

Examples of synthetic support materials are, for example, polymers such as polyamides, polyethers, polyvinyls, polyethylene, polypropylene, polytetrafluoroethylene, polyketones, polyether ketones, polyether sulfones, epoxy resins, alkyd resins, urea-aldehyde and/or melamine-aldehyde resins.

It is also possible to use glass fibers.

Preference is given to using structured supports in the form of woven metal meshes or fabrics, knitted metal meshes or fabrics or metal felts, woven carbon fibers or carbon fiber felts or woven or knitted polymer fabrics or meshes.

Monoliths made of woven materials are particularly preferred since they withstand high cross-sectional throughputs of gas and liquid and at the same time display only insignificant abrasion.

In a particularly preferred embodiment, use is made of metallic, structured supports or monoliths comprising stainless steel which preferably displays roughening of the surface when heated in air and subsequently cooled. These properties are displayed, in particular, by stainless steels in which the surface becomes enriched in an alloying constituent above a specific demixing temperature and a firmly adhering, rough oxidic surface layer is formed in the presence of oxygen as a result of oxidation. Such an alloying constituent can be, for example, aluminum or chromium from which a corresponding surface layer of Al₂O₃ or Cr₂O₃ is formed. Examples of stainless steels are those having the material numbers (in accordance with the German standard DIN 17007) 1.4767, 1.4401, 1.4301, 2.4610, 1.4765, 1.4847 and 1.4571. These steels can advantageously be thermally roughened by heating in air at from 400 to 1100° C. for from 1 hour to 20 hours and subsequently cooling to room temperature. Roughening can also be carried out mechanically in place of or in addition to thermal roughening.

Before application of the active metals and possibly promoters, the structured or monolithic supports can, if desired, be coated with one, two or more oxides. This can be carried out by physical means, for example by sputtering. Here, a thin layer of oxides, e.g. Al₂O₃, is applied to the support in an oxidizing atmosphere.

The structured supports can be shaped or rolled up, for example by means of a toothed roller, to form a monolithic catalyst element either before or after application of the active metals or promoters.

As active metals, it is in principle possible to use all metals of transition group VIII of the Periodic Table. Preference is given to using platinum, rhodium, palladium, cobalt, nickel or ruthenium or a mixture of two or more thereof, in particular ruthenium, as active metal.

Particular preference is given to using ruthenium alone as active metal. An advantage of using ruthenium as hydrogenation metal is that considerable costs can be saved in catalyst production compared to the use of the considerably more expensive hydrogenation metals platinum, palladium or rhodium.

The catalysts used for the purposes of the present invention may further comprise promoters for doping the catalyst, for example alkali metals and/or alkaline earth metals, e.g. lithium, sodium, potassium, rubidium, cesium, magnesium, calcium, strontium and barium; silicon, carbon, titanium, zirconium, tungsten and the lanthanides and actinides; coinage metals such as copper, silver and/or gold, zinc, tin, bismuth, antimony, molybdenum, tungsten and/or other promoters such as sulfur and/or selenium.

The catalysts used according to the present invention can be produced industrially by applying at least one metal of transition group VIII of the Periodic Table and, if desired, at least one promoter to one of the above-described supports.

The application of the active metals and, if desired, promoters to the above-described supports can be carried out by vaporizing the active metals under reduced pressure and condensing them continuously onto the support. Another possible method is to apply the active metals to the supports by impregnation with solutions comprising the active metals and, if desired, promoters. A further possibility is to apply the active metals and, if desired, promoters to the supports by chemical means, e.g. chemical vapor deposition (CVD).

The catalysts produced in this way can be used directly or can be heat treated and/or calcined prior to use, and can be used either in a pre-reduced state or in an unreduced state.

If desired, the support is pretreated prior to application of the active metals and, if desired, promoters. Pretreatment is advantageous when, for example, the adhesion of the active components to the support needs to be improved. Examples of pretreatment are coating of the support with adhesion promoters or roughening by mechanical methods (for instance grinding, sandblasting) or thermal methods such as heating, generally in air, plasma etching or ignition.

The present invention thus also provides structured catalyst supports to which a promoter has been applied, where the promoter is selected from among metals of the group consisting of metals of main groups I, II, IV of the Periodic Table of the Elements, metals of transition groups I to IV and VI of the Periodic Table of the Elements and sulfur, selenium and carbon, preferably structured supports. Particularly preferred promoters are: Si, Ti, Zr, Mg, Ca, C, Yt, La, Ac, Pr, W and combinations of two or more thereof.

The present invention further provides catalysts comprising a support as defined above and, applied thereto, an active metal of transition group VIII of the Periodic Table.

The preferred catalysts 1 and 2 will now be described below; as regards general features of the catalysts 1 and 2, reference is made to the above description.

### Catalyst 1

The structured support or monolith used for catalyst 1 is preferably pretreated, for example by the above-described heating in air (thermal roughening) and subsequent cooling. The support is then preferably impregnated with a solution comprising the active metal (impregnation medium). If the support is an essentially two-dimensional structured support, it can subsequently be processed to produce a monolithic catalyst element.

If the support is metallic, for example made of stainless steel, it is preferably thermally roughened by heating in air at from 400 to 1100° C. for from 1 hour to 20 hours and subsequent cooling to room temperature.

Impregnation of the support with the solution can be carried out by dipping, by allowing the solution to flow through the support or by spraying.

The impregnation medium preferably has a surface tension of not more than 50 mN/m. In a more preferred embodiment, the impregnation medium has a surface tension of not more than 40 mN/m. The minimum value of the surface tension can generally be chosen without restriction. However, in a preferred embodiment, the impregnation medium has a surface tension of at least 10 mN/m and in a particularly preferred embodiment at least 25 mN/m. The surface tension is measured by the OECD ring method known to those skilled in the art (ISO 304, cf. EC Gazette No. L 383 of Dec. 29, 1992, pages A/47-A/53).

The impregnation medium preferably comprises a solvent and/or suspension medium, for example water, in which the active metals are preferably dissolved in the form of their salts.

The impregnation medium may, if desired, further comprise promoters for doping the catalyst. In this context, reference is made to the above general description.

A solvent and/or suspension medium present in the impregnation medium is selected so that the active components, active metals, promoters or their precursors to be applied undergo no undesirable reactions therein and/or therewith.

As solvents and/or suspension media, it is possible to use the known and industrially customary solvents, for example aromatic or aliphatic hydrocarbons such as benzene, toluene, xylene, cumene, pentane, hexane, heptane, hydrocarbon fractions such as naphtha, ligroin, white oil, alcohols, diols and polyols, e.g. methanol, ethanol, the two propanol isomers, the four butanol isomers, glycol or glycerol, ethers such as diethyl ether, di-n-butyl ether, methyl tert-butyl ether, ethyl tert-butyl ether, methyl tert-amyl ether, ethyl tertamyl ether, diphenyl ether, ethylene glycol dimethyl ether, diethylene glycol dimethyl ether, triethylene glycol dimethyl ether, or water. The organic solvents or suspension media used can also be substituted, for example by halogens, e.g. chlorobenzene, or by nitro groups, e.g. nitrobenzene. The solvents or suspension media are used individually or in admixture.

Furthermore, the impregnation medium can further comprise, if necessary, auxiliaries. For example, the impregnation medium further comprises acidic or basic compounds or buffers if this is necessary or advantageous for stabilizing or solubilizing at least one of the active components or their precursors.

Preference is given to soluble salts of the active components being completely dissolved in a solvent. An aqueous solution of active components is advantageously used.

If the active composition consists of metals, particular preference is given to using either an aqueous, nitric acid solution of the nitrates of the metals or an aqueous, ammoniacal solution of amine complexes of the metals. If the active components are amorphous metal oxides, preference is given to using an aqueous sol of the oxide, which may be stabilized if desired.

The surface tension of the impregnation medium can be adjusted by means of suitable surface-active substances such as anionic or nonionic surfactants. The impregnated support is generally dried at from 100 to about 120° C. after impregnation and then, if desired, calcined at from 120 to 650° C., preferably from 120 to 400° C.

An essentially two-dimensionally structured support can, after thermal treatment, be shaped to give a three-dimensional structure having a shape appropriate to the application.

Shaping can be carried out, for example, by procedures such as cutting, corrugation of the sheets, arrangement or fixing of the corrugated sheets in the form of a monolith with parallel or crosswise channels. Shaping to give the monolith is carried out either before impregnation, before drying, before thermal treatment or before chemical treatment.

Further details of catalyst 1 and its production may be found in DE-A 198 27 385.1, whose relevant contents are fully incorporated by reference into the present application.

### Catalyst 2

The structured support or monolith used for catalyst 2 is preferably pretreated, for example by heating in air and subsequent cooling. The support is then preferably coated under reduced pressure with at least one active metal. If the support is an essentially two-dimensional structured support, it can subsequently be processed to produce a monolithic catalyst element.

Preference is given to applying not only the active metal or metals but also promoters for doping the catalyst to the support material under reduced pressure. As regards possible promoters, reference may be made to the above general description.

The support material preferably consists of metal, particularly preferably of stainless steel, more preferably stainless steels having the numbers specified earlier in the present description. The pretreatment of the support is preferably carried out by heating the metal support at from 600 to 1100° C., preferably from 800 to 1 000° C., for from 1 to 20 hours, preferably from 1 to 10 hours, in air. The support is subsequently cooled.

The active components (active metals and promoters) can be applied to the support by vapor deposition and sputtering. For this purpose, the support is coated with the active components either simultaneously or together, batchwise or continuously, at a pressure of from 10⁻³ to 10⁻⁸ mbar, preferably by means of a vapor deposition apparatus, e.g. electron beam vaporization or a sputtering apparatus. To activate the catalyst, heat treatment under inert gas or air can follow.

The active components can be applied in a plurality of layers. The catalyst obtained in this way can be processed further to produce a monolith. In this respect, reference may be made, for example, to what has been said regarding catalyst 1. The catalyst is preferably processed by shaping (corrugating, creasing) the catalyst mesh or the catalyst foil by means of a toothed roller and rolling up smooth and corrugated mesh to form a cylindrical monolith having uniform vertical channels.

Further details regarding catalyst 2 and its production may be found in EP 0,564,830, whose relevant contents are fully incorporated by reference into the present application.

### Carrying out the Process

In the process of the present invention, it is in principle possible to use all monocyclic or polycyclic aromatics which are either unsubstituted or bear one or more alkyl groups, either individually or as mixtures of two or more thereof, preferably individually. The length of the alkyl group is subject to no particular restrictions, but the alkyl groups generally have from 1 to 30, preferably from 1 to 18, in particular from 1 to 4, carbon atoms. Specific examples of suitable starting materials for the present process are, in particular, the following aromatics: benzene, toluene, xylenes, cumene, diphenylmethane, tribenzenes, tetrabenzenes, pentabenzenes and hexabenzenes, triphenylmethane, alkyl-substituted naphthalenes, naphthalene, alkyl-substituted anthracenes, anthracene, alkyl-substituted tetralins and tetralin. In the process of the present invention, preference is given to converting benzene into cyclohexane.

In the process of the present invention, the hydrogenation is preferably carried out at from about 50 to 200° C., particularly preferably from about 70 to 160° C., particularly from 80 to 100° C. The lowest temperatures can be employed especially when using ruthenium as active metal. The hydrogenation process of the present invention is preferably carried out at pressures of less than 50 bar, e.g. from 1 to 49 bar, more preferably at pressures of from 2 to 10 bar and particularly preferably at pressures of from 5 to 10 bar. As a result of the low pressures and temperatures which can be used in the process of the present invention, the formation of undesirable by-products such as methylcyclopentane or other n-paraffins is virtually nonexistent, so that complicated purification of the cycloaliphatics produced becomes unnecessary, which makes the process very economical. Despite low temperatures and pressures, the aromatic compounds can be hydrogenated in an economical manner, selectively and in a high space-time yield, to the corresponding cycloaliphatics.

The process of the present invention can be carried out either in the gas phase or in the liquid phase, with preference being given to the latter.

The process of the present invention can be carried out continuously or batchwise, preferably continuously.

The process is preferably carried out in a tubular reactor, for example a column, with product recirculation and circulating gas. Furthermore, a continuous upflow mode is preferred.

The hydrogenation according to the present invention of the aromatics is preferably carried out by passing the hydrogen-containing gas through a column provided with one of the above-described catalysts in countercurrent to the liquid aromatic or aromatics. Here, the liquid phase can be passed through the column from the top downward and the gaseous phase can be passed from the bottom upward. According to the present invention, the hydrogenation is preferably carried out continuously, in particular in countercurrent. The hydrogenation is preferably carried out in two or more stages. The catalyst described in this application is used in at least one stage. In a particularly preferred embodiment of the process of the present invention, the hydrogenation is carried out continuously in one or more reactors connected in series.

When the process is carried out continuously, the amount of the compound to be hydrogenated is preferably from about 0.05 to about 3 kg/l of catalyst per hour, more preferably from about 0.2 to about 2 kg/l of catalyst per hour.

The hydrogenation can be carried out at low cross-sectional throughput in the downflow mode, preferably in the upflow mode at high cross-sectional throughput. The cross-sectional throughputs for the liquid and gaseous phases are preferably from 150 to 600 m³/(m²·h), based on the free reactor cross section, particularly preferably from 200 to 300 m3/(m²·h). The holdup of the gas is preferably 0.5, where the holdup of the gas is defined as the quotient of the volume of gas as numerator and the sum of the volume of gas and the volume of liquid as the denominator. The pressure drop is preferably from 0.1 to 1.0 bar, particularly preferably from 0.15 to 0.3 bar, in each case per m of column height.

As hydrogenation gases, it is possible to use any gases in which free hydrogen is present and which contain no harmful amounts of catalyst poisons such as CO. For example, it is possible to use waste gases from reformers. Preference is given to using pure hydrogen as hydrogenation gas.

The hydrogenation of the present invention can be carried out in the absence or presence of a solvent or diluent, i.e. it is not necessary to carry out the hydrogenation in solution.

As solvents or diluents, it is possible to use any suitable solvent or diluent. The choice is not critical, as long as the solvent or diluent used is able to form a homogeneous solution with the aromatic to be hydrogenated.

The amount of solvent or diluent used is not restricted in any particular way and can be chosen freely according to requirements, although preference is given to using amounts which lead to a from 10 to 70% strength by weight solution of the aromatic to be hydrogenated.

When using a solvent, preference is given in the process of the present invention to using the product formed in the hydrogenation, i.e. the respective cycloaliphatic(s), as preferred solvent, if desired together with other solvents or diluents. In this case, part of the product formed in the process can be mixed into the aromatic still to be hydrogenated. Based on the weight of the aromatic to be hydrogenated, preference is given to mixing in from 1 to 30 times, particularly preferably from 5 to 20 times, in particular from 5 to 10 times, the amount of product as solvent or diluent.

In the process of the present invention, preference is given to reacting benzene at from 80 to 100° C. using ruthenium alone as active metal. A particularly preferred embodiment of the present invention, which has been found to be particularly advantageous, provides for the hydrogenation of benzene to cyclohexane to be carried out in the liquid phase in the upflow mode with product recirculation and circulating gas at a cross-sectional throughput of from 200 to 300 m³/(m²·h) at from 50° C. to 160° C. and pressures of from over a pure ruthenium/monolith catalyst. In respect of the preferred pressure and temperature ranges, what has been said above applies.

The process of the present invention has numerous advantages over the processes of the prior art. The aromatics can be hydrogenated selectively and at high space-time yield to give the corresponding cycloaliphatics at significantly lower pressures and temperatures than those described in the prior art. Even at low pressures and temperatures, the catalysts display a high activity. The cycloaliphatics are obtained in highly pure form, which makes complicated separation operations unnecessary. The formation of, for example, undesirable methylcyclopentane in the hydrogenation of benzene to cyclohexane or other n-paraffins is virtually nonexistent, so that purification of the cycloaliphatics produced becomes unnecessary. Even at low pressures, cycloaliphatics can be obtained in a high space-time yield. Furthermore, the hydrogenation can be carried out with excellent selectivity without addition of auxiliary chemicals.

The invention is illustrated by the following examples with reference to the accompanying drawing. In the drawing,

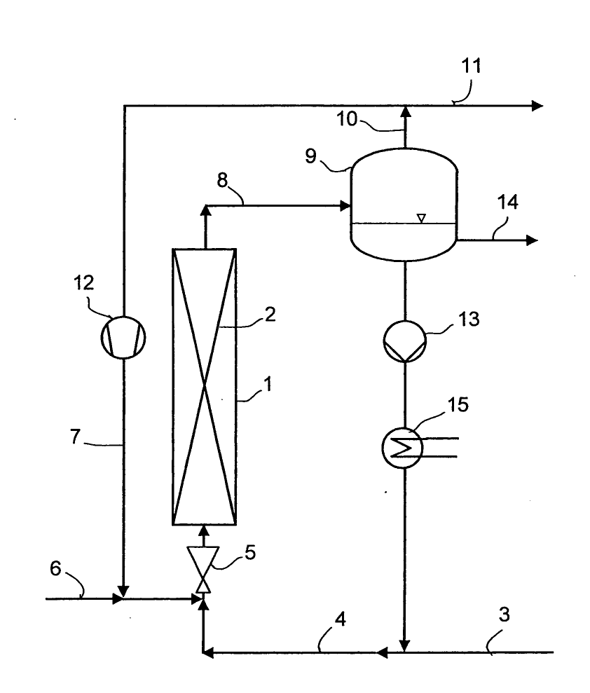

FIG. 1 shows a schematic flow diagram of a preferred embodiment of the process of the present invention.

As shown in FIG. 1, the process of the present invention can be carried out in a tubular reactor 1, for example a column, with product recirculation and circulating gas. FIG. 1 shows a continuous upflow mode of operation using a packed bubble column. A monolithic catalyst 2 is installed as a fixed bed in the reactor 1. Feed via feed line 3 together with circulating liquid are fed via line 4 as driving jet into a mixing nozzle 5 in which fresh hydrogen via line 6 and circulating gas via line 7 are mixed in. The two-phase gas/liquid mixture 8 leaves the reactor 1 at its upper end and is separated in a gas/liquid separator 9. A substream 11 of the gas stream 10 is discharged. The circulating gas stream 7 is recirculated via a compressor 12 to the mixing nozzle 5. This compressor 12 can, if desired, be omitted if the circulating liquid 4 which is conveyed via the pump 13 can be supplied at sufficiently high pressure and the mixing nozzle 5 is designed as a jet compressor. A substream 14 is taken as product stream from the circulating liquid 4. The volume ratio of circulating liquid 5 to product stream 14 is from 90:1 to 500:1, preferably from 150:1 to 250:1. Heat exchange is regulated by means of the heat exchanger 15. The diameter of the tube reactor 1 is designed so that an empty tube velocity of the liquid of from 100 to 1000 m/h results.

### EXAMPLES OF PRODUCTION OF CATALYSTS

### Catalyst Production Example 1

This monolithic catalyst was produced from a woven V2 A strip, material No. 1.4301, which was coated with 0.455 g of Ru/m² and had previously been ignited in air for 3 hours at 800° C. This woven strip had been coated by impregnation with a ruthenium salt solution. The coated woven mesh was subsequently heated at 200° C. for 1 hour. 51 cm of the 20 cm wide catalyst mesh strip were corrugated by means of a toothed roller, modulus 1.0 mm, and rolled up together with a 47 cm long smooth catalyst mesh strip so as to form a monolith having vertical channels and a diameter of 2.7 cm (catalyst A).

### Catalyst production example 2

This monolithic catalyst was produced from a woven V2 A strip, material No. 1.4301, coated with 0.432 g of Ru/m². The woven strip was ignited in air at 800° C. for 3 hours, after which 2000 Å of silicon were applied to it by vapor deposition. The silicon-coated woven strip was subsequently heated at 650° C. This woven strip was subsequently coated with a total of 0.432 g of Ru/m² by impregnation with a ruthenium salt solution. The coated mesh strip was subsequently heated at 200° C. for 1 hour. 51 cm of the 20 cm wide catalyst mesh strip were corrugated by means of a toothed roller, modulus 1.0 mm, and rolled up together with a 47 cm long smooth catalyst mesh strip so as to form a monolith having vertical channels and a diameter of 2.7 cm (catalyst B).

### PROCESS EXAMPLES

### Process Example 1

Three monolithic ruthenium catalysts A having a total volume of 343 cm³ were installed in a heatable double-walled tube reactor. The apparatus was subsequently flushed with N₂ and the N₂ was then replaced by H₂ and the catalyst was reduced for 1 hour at 80° C. It was subsequently cooled and the circuit of the plant was supplied with benzene. Hydrogenation was carried out at 100° C., 8 bar and a cross-sectional throughput of liquid and gas of 200 m³/(m²·h) using the process flow indicated in FIG. 1.

GC analyses of the reaction product showed quantitative conversion of benzene and a yield of 99.99%. The space-time yield was 0.928 kg/(l·h). Methylcyclopentane could not be detected.

### Process Example 2

Three monolithic ruthenium catalysts B having a total volume of 343 cm³ were installed in a heatable double-walled tube reactor. The apparatus was subsequently flushed with N₂ and the catalyst was not prereduced. The circuit of the plant was subsequently supplied with benzene and hydrogen was injected. Hydrogenation was carried out at 100° C., 8 bar and a cross-sectional throughput of liquid and gas of 200 m³/(m²·h) using the process flow indicated in FIG. 1.

GC analyses of the reaction product showed quantitative conversion of benzene and a yield of 99.99%. The space-time yield was 0.802 kg/(l·h). Methylcyclopentane could not be detected.

We claim:

1. A process for the hydrogenation of at least one monocyclic or polycyclic aromatic which may be unsubstituted or substituted by at least one alkyl group by bringing the aromatic into contact with a hydrogen-containing gas in the presence of a catalyst comprising at least one metal of transition group VIII of the Periodic Table as active metal applied to a structured or monolithic support.

2. A process as claimed in claim 1, wherein the hydrogenation is carried out at pressures of less than 50 bar, preferably at pressures of from 5 to 10 bar.

3. A process as claimed in claim 1 or 2, wherein the structured support is selected from among woven fabrics and meshes, knitteds, felts, films and foils, metal sheets and expanded metal.

4. A process as claimed in any of the preceding claims, wherein the support comprises metallic, inorganic, organic or synthetic materials or combinations of such materials.

5. A process as claimed in any of the preceding claims, wherein ruthenium alone is used as active metal.

6. A process as claimed in any of the preceding claims, wherein a supported catalyst obtainable by heating the structured support or monolith in air and cooling, subsequent impregnation with a solution comprising the active metal or metals, and, if desired, processing to form a monolithic catalyst element is used.

7. A process as claimed in any of claims 1 to 5, wherein a supported catalyst obtainable by heating the structured support or monolith in air and cooling, subsequent coating with the active metal or metals under reduced pressure and, if desired, processing to form a monolithic catalyst element is used.

8. A process as claimed in any of the preceding claims, wherein benzene is hydrogenated to cyclohexane or aniline is hydrogenated to cyclohexylamine.

9. A process as claimed in any of the preceding claims, wherein the hydrogenation is carried out at from 70 to 160° C.

10. A process as claimed in any of the preceding claims, wherein benzene is hydrogenated at from 80 to 100° C. and ruthenium alone is used as active metal.

11. A process as claimed in any of the preceding claims, wherein the hydrogenation is carried out continuously and in countercurrent.

12. A structured catalyst support to which a promoter has been applied, wherein the promoter is selected from among metals of main groups I, II, IV of the Periodic Table of the Elements, metals of transition groups I to IV and VI of the Periodic Table of the Elements and sulfur, selenium and carbon.

13. A structured support as claimed in claim 12, wherein the promoter is selected from the group consisting of: Si, Ti, Zr, Mg, Ca, C, Yt, La, Ac, Pr, W and combinations of two or more thereof.

14. A structured support as claimed in claim 12 or 13, wherein the support material has a surface which has been roughened by thermal, chemical or thermal and chemical treatment.

15. A catalyst comprising a support as claimed in any of claims 12 to 14 and, applied thereto, an active metal of transition group VIII of the Periodic Table.
